(19) United States
(12) Patent Application Publication
(10) Pub. No.: US 2014/0288015 A1
(43) Pub. Date: Sep. 25, 2014

(54) METHODS AND DEVICES FOR AFFECTING NERVE FUNCTION

(71) Applicants: Kondapavulur T. VENKATESWARA-RAO, San Jose, CA (US); Emily A. STEIN, San Leandro, CA (US); Michael A. EVANS, Palo Alto, CA (US); Mark H. WHOLEY, Oakmont, PA (US)

(72) Inventors: Kondapavulur T. VENKATESWARA-RAO, San Jose, CA (US); Emily A. STEIN, San Leandro, CA (US); Michael A. EVANS, Palo Alto, CA (US); Mark H. WHOLEY, Oakmont, PA (US)

(21) Appl. No.: 14/217,109

(22) Filed: Mar. 17, 2014

Related U.S. Application Data
(60) Provisional application No. 61/794,763, filed on Mar. 15, 2013.

(51) Int. Cl. A61K 31/7048; A61K 31/70

(52) U.S. Cl. CPC A61K 31/7048; A61K 31/70
USPC 514/26; 604/506; 514/23

(57) ABSTRACT

Various methods and devices are described for affecting nerve function in the carotid body, renal nerves, and other nerves. Syringes, endovascular catheters, drug-eluting balloons, drug-eluting stents, and agent delivery patches are used to deliver a neuromodulatory agent to one or more nerves in order to treat a disease state.

### CROSS-REFERENCE TO RELATED APPLICATION

This application claims the benefit of U.S. provisional patent application Ser. No. 61/794,763, filed Mar. 15, 2013, which is hereby incorporated by reference in its entirety.

### BACKGROUND

Recent studies have demonstrated that the sympathetic nervous system also plays a significant role in influencing human disease. Much like atherosclerosis in blood vessels, where plaque leads to constriction of blood flow and myocardial infarction, the human nervous system also becomes diseased or dysfunctional with age. Different nerves (afferent and efferent), receptors and nerve plexi inside the body become abnormal or imbalanced in terms of overactivity, hypersensitivity to chemosensory stimuli and elevated sympathoexcitatory response to peripheral chemoreceptor stimulation. Specifically, it has been shown that overactivity of the sympathetic nervous system and enhanced peripheral chemoreflex sensitivity is linked with hypertension and heart failure. New technologies, like radiofrequency ablation, ultrasound ablation, cryo-ablation, and chemo-ablation, are being developed to reduce this overactivity and hypersensitivity which can lead to new therapies for treating disease.

Optimizing the sympathetic nerve activity can prevent hypertension and insulin resistance (incidence and control of Type II diabetes). It can reduce symptoms of SDB (Obstructive Sleep Apnea and CSA), tachy arrhythmias (Atrial Fibrillation or AFib and Ventricular tachycardia-VT) PCO and fertility. It can also reduce morbidity and mortality by treating heart failure (prevention of ADHF, cardiorenal syndromes), chronic kidney disease (CKD) and end-stage renal disease (ESRD).

Insulin Resistance: Diabetes and metabolic syndrome. Sympathetic activity mediates vascular resistance. Blood flow is shifted from striated muscle (insulin sensitive) to visceral tissue (insulin resistant). Sympathetic neural activity (measured as impulses/100 beats) is significantly high in diabetic and hypertensive patients and patients suffering from both.

Other conditions include sexual dysfunction (ED, PE), pulmonary—COPD, and other, e.g. obesity, dyslipidaemia.

Energy-based approaches using cryo, radiofrequency ablation and ultrasound are not capable of selectively targeting neurons and can damage surrounding tissue, such as smooth muscle cells in the intima and media of blood vessels.

Implants—Implantable (electrical-stimulation) generators are expensive and require invasive procedures. Implants like arterial-venous fistulas reduce peripheral blood circulation. Other implants may cause damage to tissue and impede blood flow in the long-term (stenosis).

What is needed are methods and devices that overcome these limitations. Devices are designed intended to access specific nerve locations inside the body and methods are described for locally delivering neurotropic agents that affect neurons and neuronal function and treat specific disease states.

### BRIEF DESCRIPTION OF THE DRAWINGS

FIG. 1A shows the sympathetic and parasympathetic nervous systems.

FIG. 1B shows the carotid body and surrounding anatomy.

FIGS. 1C-1D show examples of dose-dependent mechanisms of action of an agent in denervation and nerve modulation, respectively.

FIG. 2 shows one embodiment of a syringe 100.

FIGS. 3A-3F show one embodiment of a method for using syringe 100 to directly access the carotid body and deliver an agent to the carotid body.

FIGS. 3G-3L show one embodiment of a method for using syringe 100 to directly access the renal arteries and deliver an agent to the renal nerves.

FIGS. 4A-4B show side and cross-sectional views, respectively, of one embodiment of an endovascular catheter 200.

FIGS. 5A-5E show one embodiment of a method for using endovascular catheter 200 to deliver an agent to the wall of the renal artery.

FIG. 6 shows one embodiment of a balloon 300.

FIGS. 7A-7D show one embodiment of a method for using balloon 300 to deliver an agent to the carotid body.

FIG. 8 shows one embodiment of a stent 400.

FIGS. 9A-9B show one embodiment of a method for using stent 400.

FIG. 10 shows one embodiment of an agent delivery patch 500.

FIG. 11 shows one embodiment of a method for using drug patch 500 to deliver an agent to the carotid body.

### DESCRIPTION

It has been shown that reduction in the renal sympathetic nerve activity (afferent and/or efferent) through renal denervation is desirable to treat resistant hypertension. Current systems may not provide immediate feedback to the physician when denervation is complete or whether some or all of the nerves are destroyed, and whether the treatment is effective or not. In other words, current technologies may not be able to measure the optimal dose of denervation to treat patients. Some patients continue to remain resistant to treatment after multiple RF ablation treatments. The optimal dose of afferent and efferent denervation is not known and may be different for different patient populations, disease states and clinical endpoints. In some cases complete denervation may not be necessary.

For example, denervation to reduce overactivity in RSNA may be different for different patients. And with the same patient the denervation dose may be different to treat tachy arrhythmias or atrial fibrillation (AF in the heart) compared to treatment of COPD (denervation of the pulmonary nerves) and treatment of obesity (vagus nerve denervation). Feedback is useful to verify that treatment is complete. Methods and systems are described to measure SNA pre, peri and post treatment to ensure that optimal treatment is delivered.

For example, the local delivery of a neurotropic agent into the renal artery and acting on renal nerves to treat resistant hypertension is described in U.S. patent application Ser. No. 13/014,700 (filed Jan. 26, 2011), Ser. No. 13/014,702 (filed Jan. 26, 2011), Ser. No. 13/096,446 (filed Apr. 28, 2011), 61/551,921 (filed Oct. 26, 2011), and 61/644,134 (filed May 8, 2012), each of which are hereby incorporated by reference. In the present application, methods and devices are described to treat other disease states by affecting neurons and neuronal pathways at other locations inside the human body.

Agents include channel blockers, neuronal antagonistic monoclonal antibodies such as anti-nerve growth factor and anti-norepinephrine, nerve toxins such as BOTOX, conotoxin, ion pump blockers, vasodilators, and vasoconstrictors.

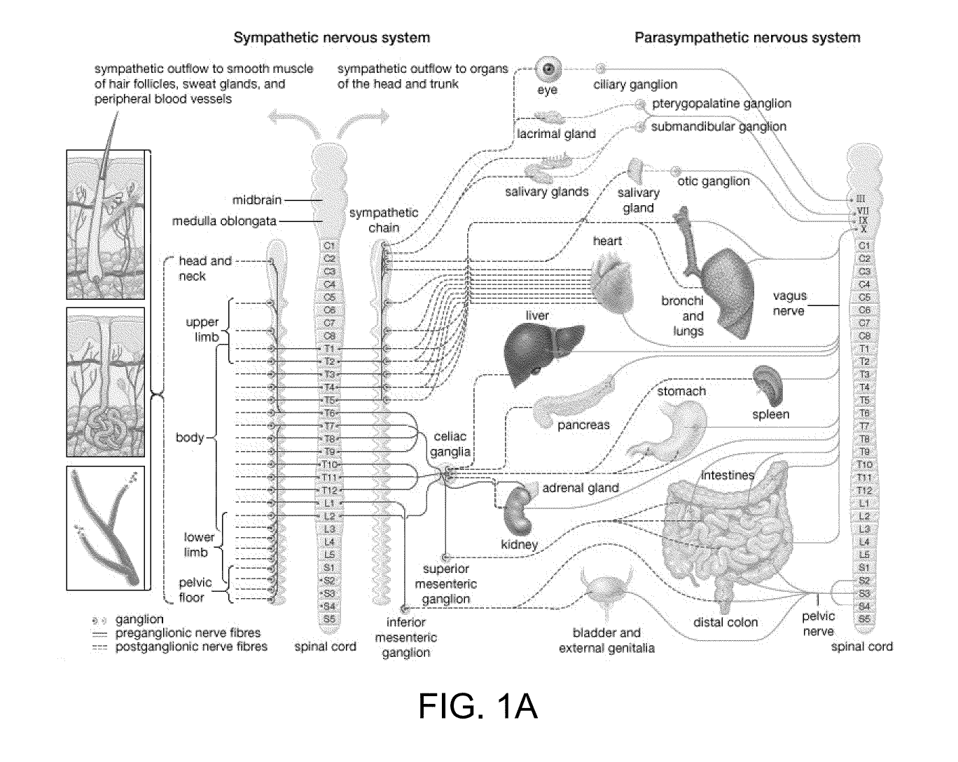

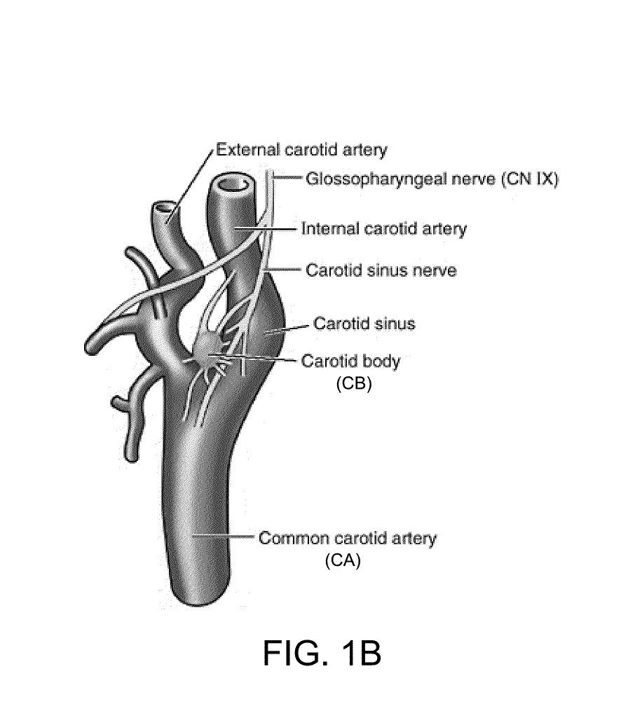

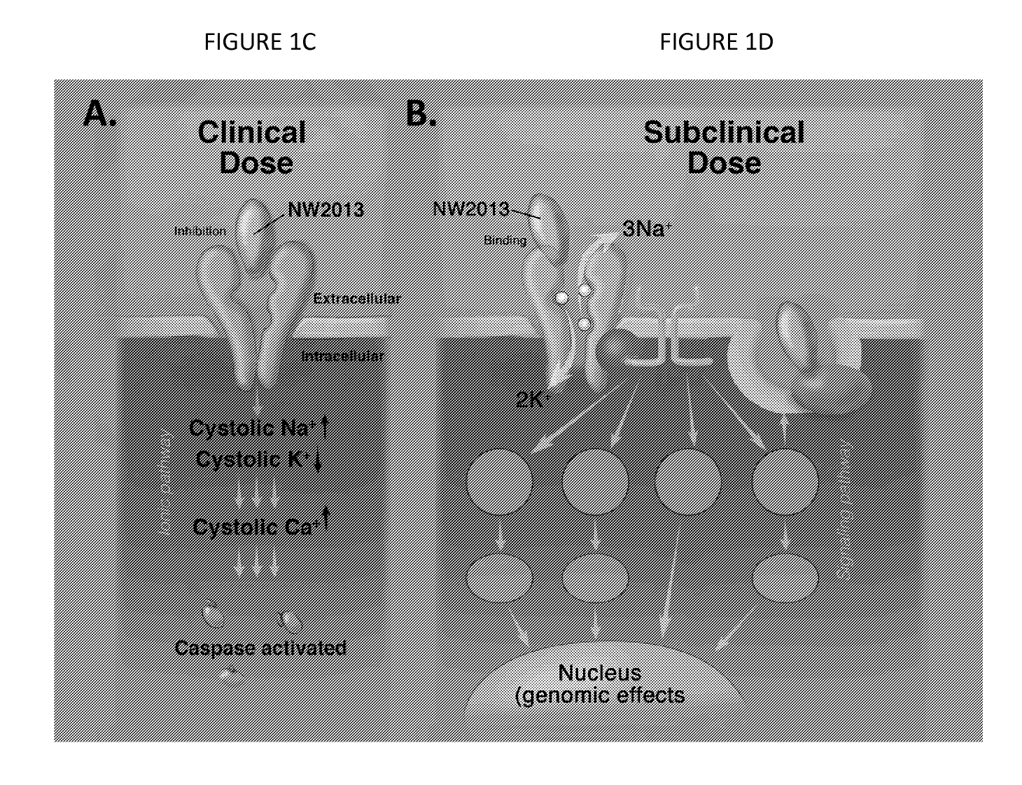

FIG. 1A shows the sympathetic and parasympathetic nervous systems. Several targets are shown here, including the vagus nerve, pulmonary nerves, neural pathways that cause atrial fibrillation, and others. The carotid body may be targeted to treat hypertension and other disease states. The renal arteries may be targeted to treat hypertension and other disease states. Neural pathways associated with the pulmonary veins may be targeted to treat atrial fibrillation. The celiac axis plexus may be targeted to treat pain caused by cancer and other disease states.

FIG. 1B shows the carotid body CB and surrounding anatomy. The carotid body CB is located at the carotid artery bifurcation and contains baroreceptors and chemoreceptors. The carotid body CB is adrenergic excitatory. It increases central sympathetic outflow, inhibits parasympathetic outflow and increased organ-specific adrenergic activity (muscle, vascular, renal and cardiac sympathetic nerve activity (SNA)). Thus, treating the carotid body CB can reduce adrenergic hyperactivity of SNA and related pathological changes in muscles, blood vessels, kidney and the heart. Treatment of the carotid body CB may modulate and suppress overactivity.

FIGS. 1C-1D show examples of dose-dependent mechanisms of action of an agent in denervation and nerve modulation, respectively. FIG. 1C shows an example of how the dose of agent delivered may be high to induce the activation of death-promoting signaling machinery such as caspases to result in denervation. FIG. 1D shows how the dose of agent delivered may be lower to induce the activation of signaling machinery that promotes a down-regulation in nerve metabolism, nerve impulse generation, neurotransmission, nerve firing frequency, or other to result in nerve modulation.

FIG. 2 shows one embodiment of a syringe 100. Syringe 100 may be used to directly access and inject an agent into and/or around the carotid body CB and other targets. Syringe 100 includes a chamber 101, a plunger 102 slidably disposed within chamber 101, and a needle 103 having a lumen 104 in fluid communication with chamber 101. Chamber 101 may be configured to contain an agent. Needle 103 may have a tip 105 that is sharp. Needle 103 may include one or more needle markers 106. Needle markers 106 may be radiopaque to aid in visualization. Needle 103 may include one or more side holes 107 in fluid communication with lumen 104. Side holes 107 may be configured to deliver a drug horizontally following the tissue planes of an artery or other structure/vessel. Needle 103 may be treated with or made of a material that may be visualized by ultrasound or other imaging methods. Needle 103 may be made of a polymer, a metal, or any other suitable material.

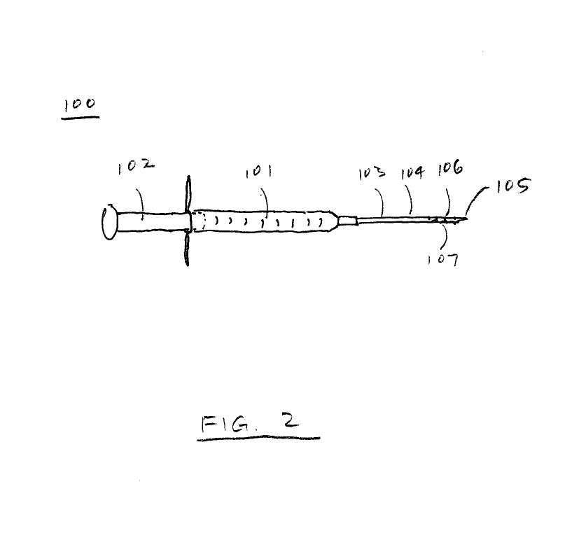

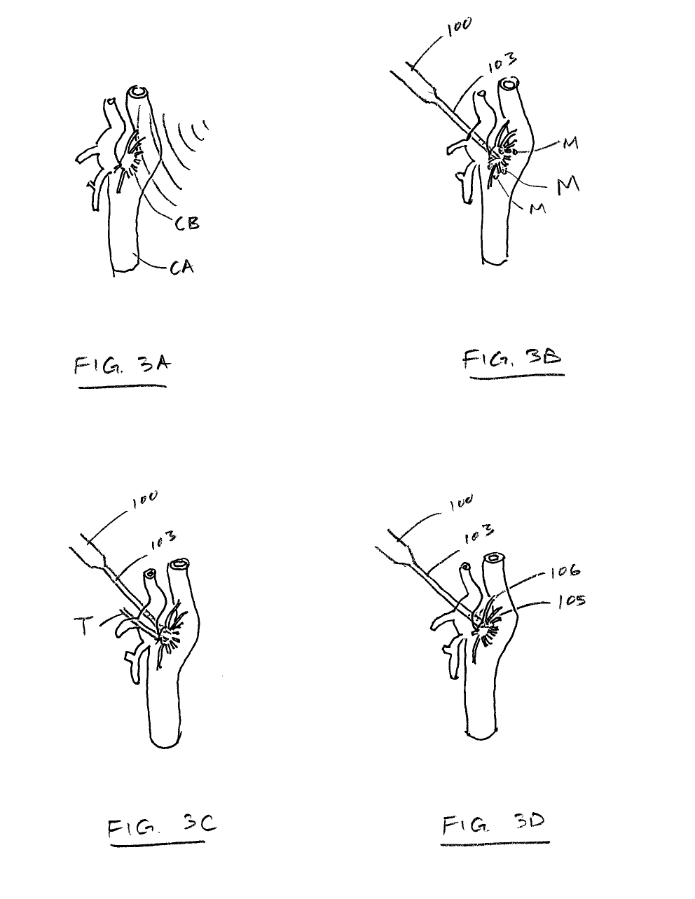

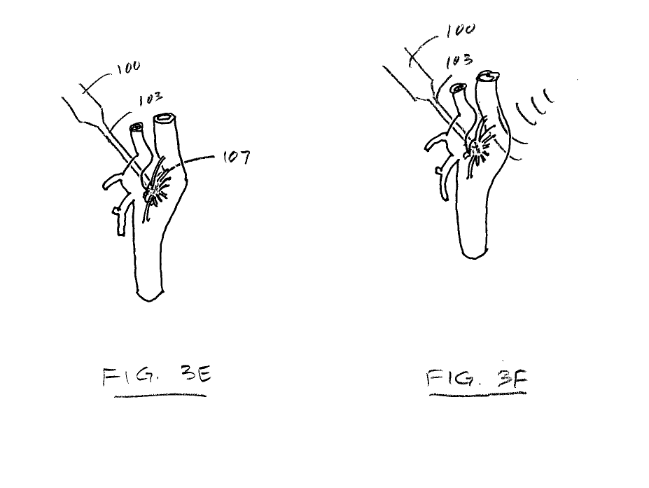

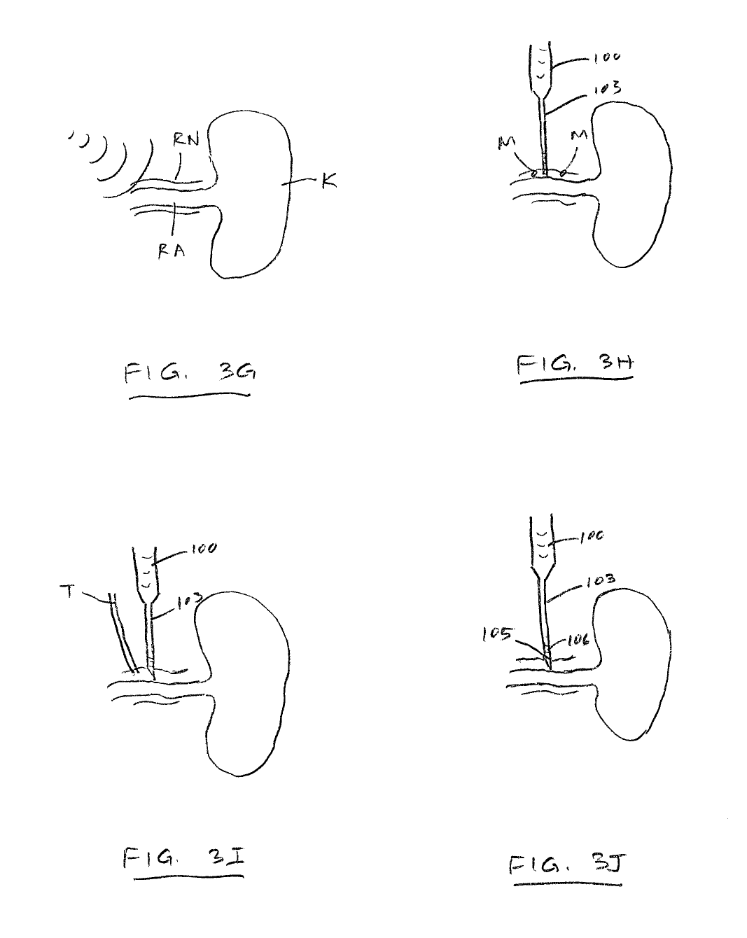

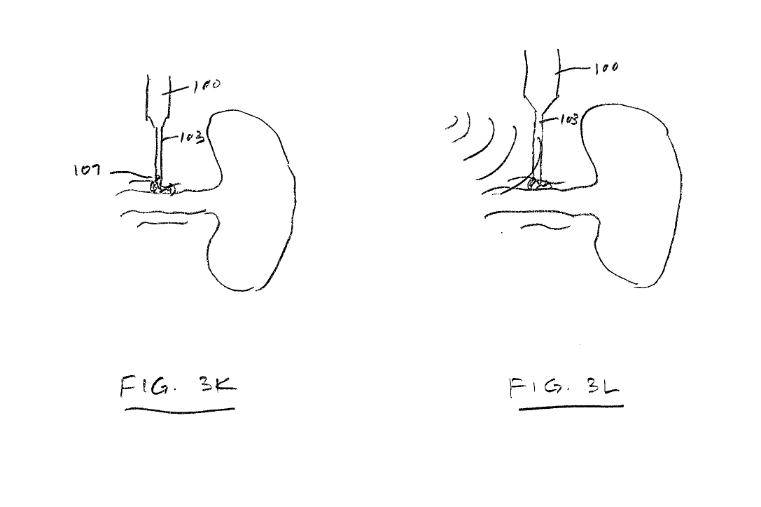

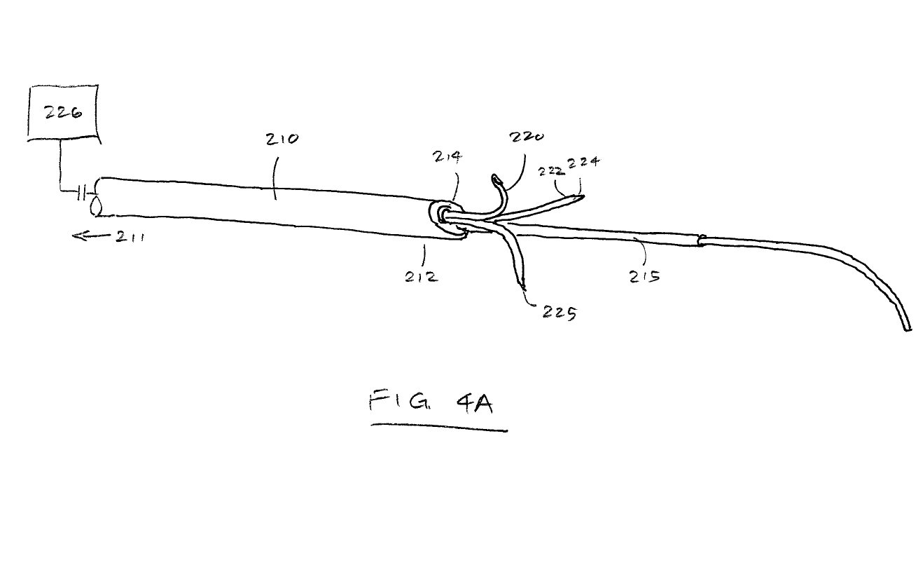

FIGS. 3A-3F show one embodiment of a method for using syringe 100 to directly access the carotid body CB and deliver an agent to the carotid body CB. FIG. 3A shows locating the carotid body CB. The carotid body CB may be located with the aid of CT 3-D reconstruction. Ultrasound may also be used in combination with CT. MRI, OCT, and/or other imaging methods may also be used. The carotid body CB may be accessed from the angle of the mandible to the carotid body/bifurcation. FIG. 3B shows placing one or more small relocation markers M into or around the carotid body CB. The relocation markers M may be placed using the same or a different syringe 100, or other surgical methods. The relocation markers M may be radiopaque. The relocation markers M may facilitate relocation of the carotid body CB for subsequent treatment. Alternatively, or in addition, a small, flexible tube T may be placed into or around the carotid body CB, as shown in FIG. 3C. The tube T may be radiopaque. The tube T may facilitate access to the carotid body CB for subsequent treatment.

FIG. 3D shows placing needle 103 into or around the carotid body CB. For example, tip 105 of needle 103 may be placed near or at an external surface of the carotid artery CA. Needle markers 106 may be used to help confirm proper positioning in or around the carotid body CB. FIG. 3E shows injecting an agent into and/or around the carotid body CB. Side holes 107 may allow the agent to be distributed horizontally following the tissue planes of the carotid artery. This may allow for greater distribution of the agent along the fascial plane of the adventitia. FIG. 3F shows verifying or validating the injection of agent into and/or around the carotid body CB. A visualizable agent may be used. For example, the agent may include contrast, microspheres, and/or microbubbles to enhance visualization.

FIGS. 3G-3L show one embodiment of a method for using syringe 100 to directly access the renal artery RA and deliver an agent to the renal nerves RN. FIG. 3G shows locating the renal artery RA leading to the kidney K. The renal nerves RN are located in the wall of the renal artery RA. The renal artery CB may be located with the aid of CT 3-D reconstruction. Ultrasound may also be used in combination with CT. MRI, OCT, and/or other imaging methods may also be used. The renal artery RA may be accessed from the dorsal side, adjacent to the spine, to the renal perivascular space. FIG. 3H shows placing one or more small relocation markers M into or around the wall of the renal artery RA. The relocation markers M may be placed using the same or a different syringe 100, or other surgical methods. The relocation markers M may be radiopaque. The relocation markers M may facilitate relocation of the renal artery RA for subsequent treatment. Alternatively, or in addition, a small, flexible tube T may be placed into or around the wall of the renal artery RA, as shown in FIG. 3I. The tube T may be radiopaque. The tube T may facilitate access to the renal artery RA for subsequent treatment.

FIG. 3J shows placing needle 103 into or around the wall of the renal artery RA. Needle markers 106 may be used to help confirm proper positioning in or around the wall of the renal artery RA. FIG. 3K shows injecting an agent into and/or around the wall of the renal artery RA. Side holes 107 may allow the agent to be distributed horizontally following the tissue planes of the renal artery. This may allow for greater distribution of the agent along the fascial plane of the adventitia. FIG. 3L shows verifying or validating the injection of agent into and/or around the wall of the renal artery RA. A visualizable agent may be used. For example, the agent may include contrast, microspheres, and/or microbubbles to enhance visualization.

Similar methods may be used to locally deliver agents to other nerve plexi or target nerve tissue in the human body to restore sympathetic balance or sympathetic tone and reduce overactivity, as detailed in FIG. 1A, to treat diseases or conditions caused by a degenerated nervous system. Examples include delivering the agents into the heart muscle to affect nerve function and treat cardiac arrhythmias.

The majority of the renal sympathetic nerves are near the lumen-intima interface in HTN patients compared to Normal patients (accessible to catheter ablation). Significant increase in afferent axons compared to efferent in HTN compared to Normal suggesting increased sympathetic activity. No difference in the polar and longitudinal distribution of sympathetic nerve fibers between groups. Other studies have shown that the average nerve distance is about 3.2 mm from the endothelium. Efficient treatment must account for this large distance and variabilities in nerve distribution between patients.

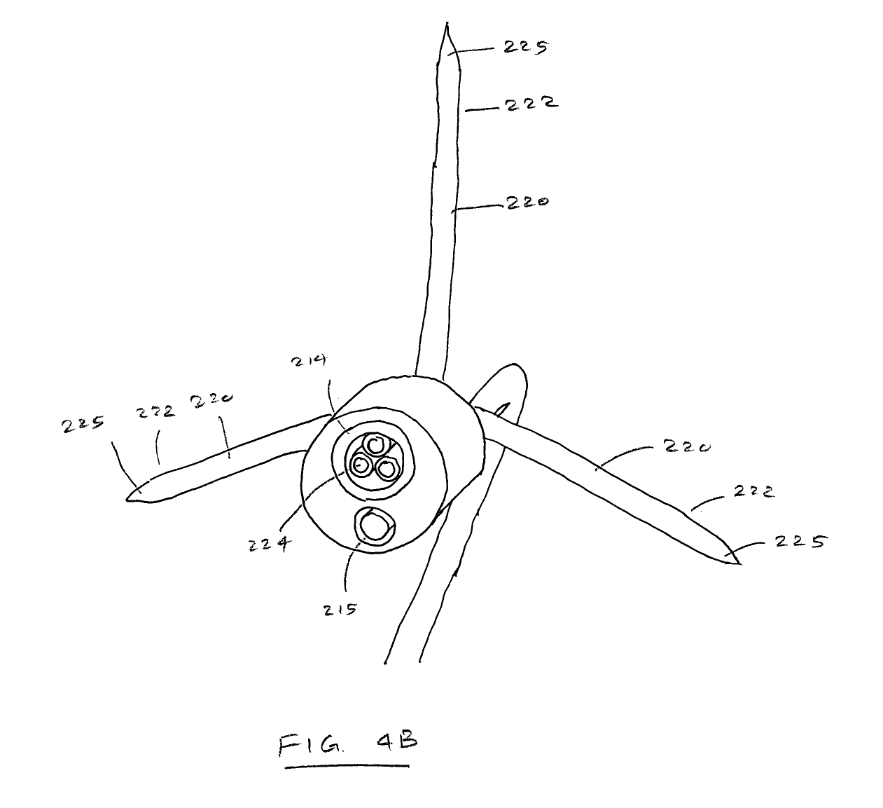

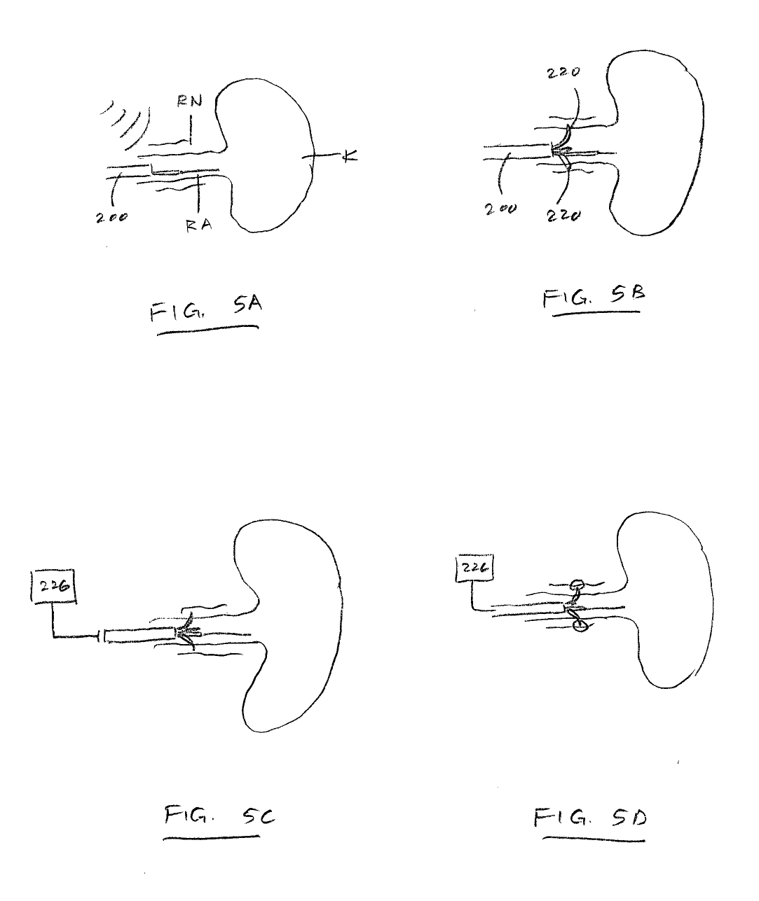

FIGS. 4A-4B show side and cross-sectional views, respectively, of one embodiment of an endovascular catheter 200. Endovascular catheter 200 may include a body 210 and one or more microneedles 220. Body 210 may include a proximal portion 211, a distal portion 212, a working lumen 214, and a guidewire lumen 215. Microneedles 220 may be slidably disposed within working lumen 214. Microneedles 220 may be self-expanding when extended from working lumen 214. Microneedles 220 may include a distal portion 222 and a needle lumen 224. Microneedles 220 may include tips 225 that are sharp. Microneedles 220 may be coupled to electronics 226 which allow microneedles 220 to be used as sensors for monitoring nerve activity through electrical conductance measurements or microneurography. Guidewire lumen 215 may include an extension from distal portion 212 of body 210.

Microneedles 220 may be coated and/or mechanically textured to enhance visibility under ultrasound, CT, MRI, and/or other imaging methods. Microneedles 220 may be made from materials and coatings that have good combination of electrical conductivity, mechanical strength, and biocompatibility. Conductive coatings may include metallic coatings of gold, platinum, iridium, tungsten, and/or silver on stainless steel or NITINOL needles.

Coatings may include conducting polymer black coatings on stainless steel and NITINOL-like poly(acetylene)s, polyaniline, polythiophene and polypyrrole (doped with iodine, bromine and chlorine) Poly(3,4-ethylenedioxythiophene) or PEDOT, PEDOT:PSS (polystyrene sulfonic acid) dispersions. Coatings may include conducting polymer nanocomposite sensors of carbon black and polyaniline. Microneedles 220 may be made of MP35N, L605 cobalt chromium, and tungsten alloys.

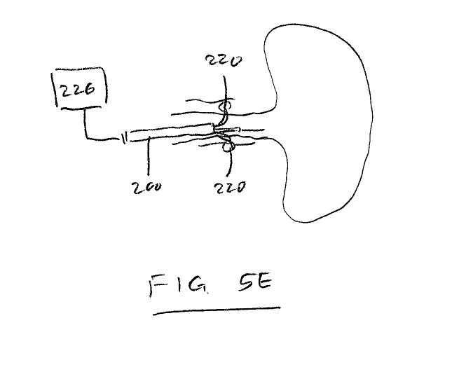

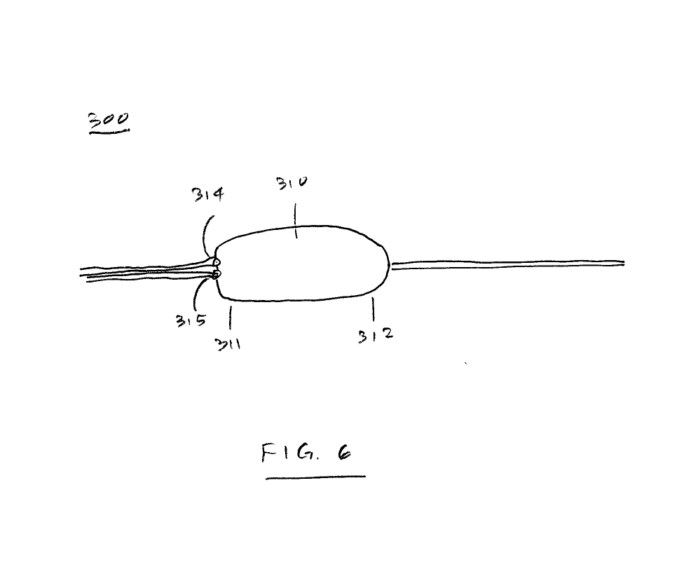

FIGS. 5A-5E show one embodiment of a method for using endovascular catheter 200 to deliver an agent to the wall of the renal artery RA. FIG. 5A shows positioning endovascular catheter 200 within the renal artery RA leading to the kidney K. Optical, OCT, CT, MRI, ultrasound, or any other suitable imaging method may be used. The renal nerves RN are located in the wall of the renal artery RA. FIG. 5B shows extending microneedles 220 out of working lumen 214 and into the wall of the renal artery RA. Microneedles 220 may be used as sensors to confirm location of the renal nerves RN. Optical, OCT, CT, MRI, ultrasound, or any other suitable imaging method may be used. FIG. 5C shows assessing sympathetic nerve activity or overactivity to diagnose a disease. Microneedles 220 may be used as sensors with electronics 226. FIG. 5D shows injecting the agent into wall of the renal artery RA, while simultaneously measuring changes in electrical activity of the renal nerves. A drop or rise in nerve signals at low, medium, and high frequencies may be measured. Microneedles 220 may be used as sensors with electronics 226. FIG. 5E shows adjusting the amount of agent delivered into the wall of the renal artery RA, based on the nerve signal feedback.

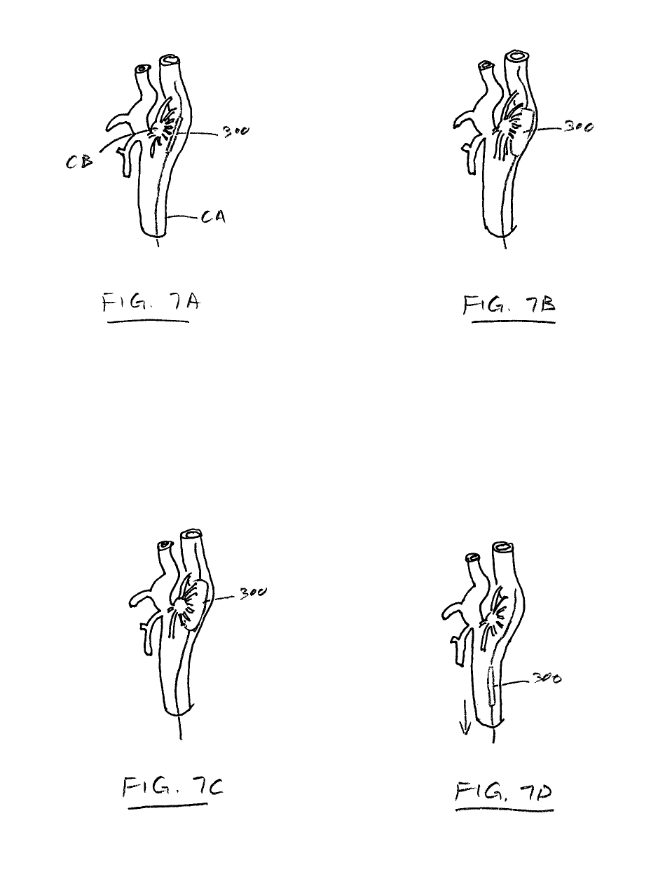

FIG. 6 shows one embodiment of a balloon 300. Balloon 300 may include a body 310. Body 310 may include a proximal portion 311, a distal portion 312, an inflation lumen 314, and a guidewire lumen 315. Balloon 300 may be drug-eluting. Balloon 300 may be coated with one or more agents. Balloon 300 may be coated with carrier molecules such as liposomes, cholesterol, arachidonic acid, propylene glycol, linoleic acid, and/or oleic acid. Balloon 300 may be configured to be expanded at a treatment site within a vessel, and held in place for a desired period of time to deliver an agent into the wall of the vessel. Balloon 300 may optionally include one or more microneedles coupled to an exterior surface of balloon 300 to enhance delivery of an agent.

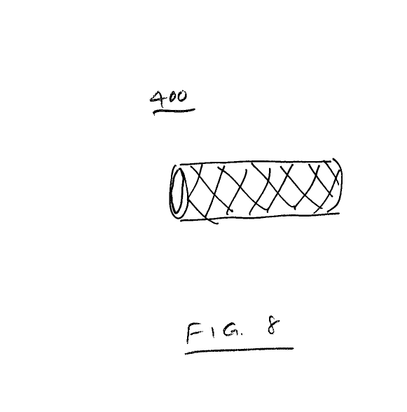

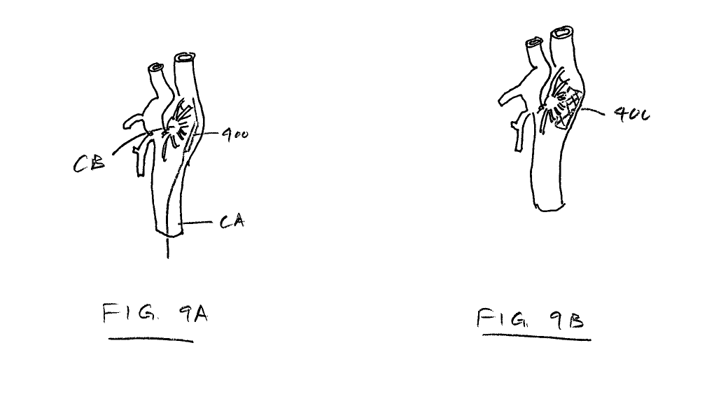

FIGS. 7A-7D show one embodiment of a method for using balloon 300 to deliver an agent to the carotid body. FIG. 7A shows positioning balloon 300 in the carotid artery in a vicinity of the carotid body. Ultrasound, 3-D CT, or any other imaging method may be used. FIG. 7B shows expanding balloon 300 to bring balloon 300 in contact with the walls of the carotid artery. FIG. 7C shows holding balloon 300 inflated for a fixed period of time in the carotid artery. Balloon 300 may deliver an agent to the carotid baroreceptor plexus. FIG. 7D shows deflating balloon 300 and removing balloon 300 from the carotid artery.

FIG. 8 shows one embodiment of a stent 400. Stent 400 may be drug-eluting. Stent 400 may be coated with one or more agents. Stent 400 may be biostable and/or bioabsorbable. Stent 400 may be configured to enhance the vaso-elastic properties (compliance) of arteries and improve blood flow and vascular resistance by eluting neurotropic agents that, affect nerve function and treat hypertension, CHF, and other disease states, and affect nerve function and improve vasodilation properties of the blood vessel and reduce arterial stiffness. Stent 400 may be configured to affect inflammation and treat cardiovascular disease (neurotropic agent also has anti-inflammatory properties) by incorporating the drug as a single agent, in combination with other neurotropic agents, or in combination with other anti-inflammatory compounds such as sirolimus, everolimus, zotarolimus, and others.

Stent 400 may be configured to act on chemoreceptors (electrical), chemosensors, and plaque stabilizers (known to scavenge foam cells). Stent 400 may be configured to affect ACS and SCD.

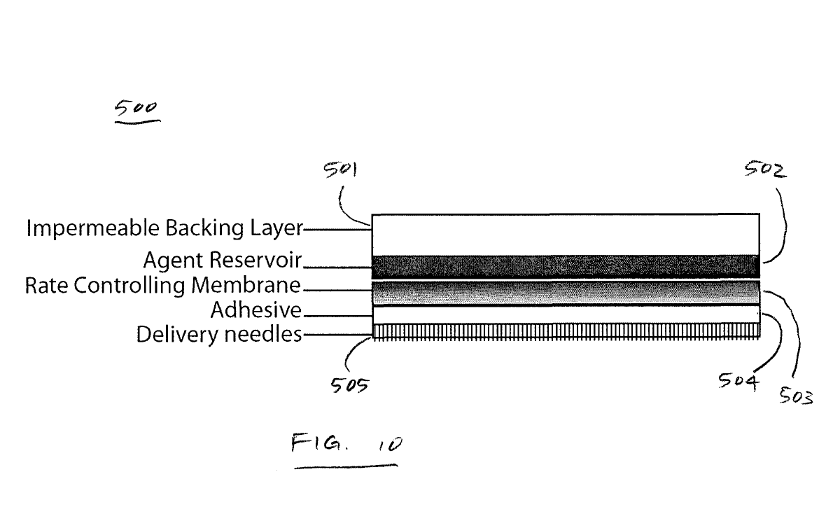

FIGS. 9A-9B show one embodiment of a method for using stent 400. FIG. 9A shows stent 400 positioned at a treatment site within a vessel, such as within the carotid artery to treat the carotid body. Stent 400 may be delivered using a balloon catheter. CT, MRI, ultrasound, or any other imaging method may be used. FIG. 9B shows stent 400 being expanded at the treatment site. Stent 400 may elute a drug into the wall of the vessel.

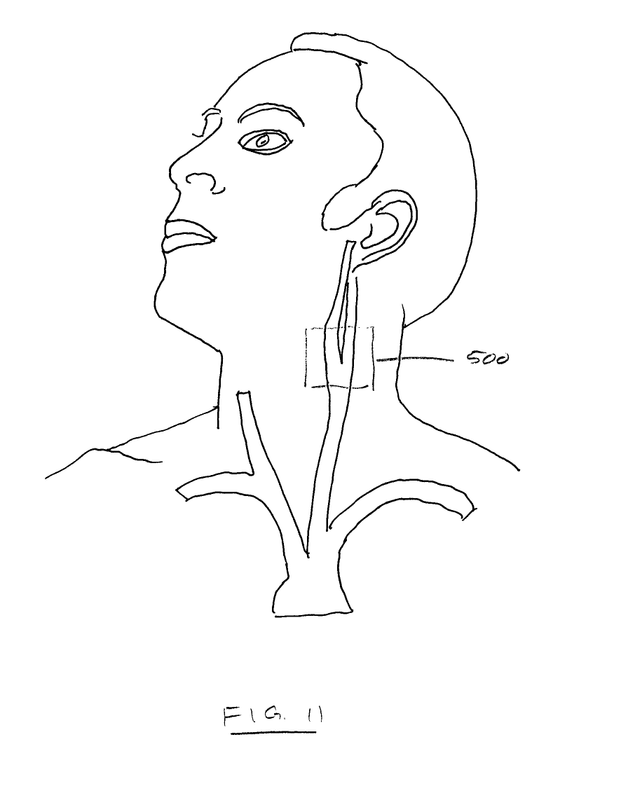

FIG. 10 shows one embodiment of an agent delivery patch 500. Agent delivery patch 500 may include a protective impermeable backing layer 501 to inhibit the loss of agent from the exterior of the patch, wherein said layer is hydrophilic in nature. Next to impermeable backing layer 501 may be an agent reservoir layer 502 comprising a polymeric hydrophilic matrix with pores containing agent. Next to the agent reservoir layer 502 may be a rate controlling membrane 503 comprising a polymeric matrix having pores of varying diameters, wherein larger pores are closest to agent reservoir 502 and smallest pores are closest to an adhesive layer 504, which serves to modulate agent diffusion into adhesive layer 504 and delivery needles 505. Adhesive layer 504 may include an adherent material capable of binding to the epidermal layer for 24-48 hours and may include a structural support system to maximize contact of delivery needles 505 with the skin. Delivery needles 505 may include hydrophilic polymers to deliver agent into the dermis of the skin. Upon application of agent delivery patch 500, agent diffuses radially away from the injection site at physiologic rates. Agents that may be used in a agent delivery patch 500 for the purpose of nerve modulation may include neurotropic properties and diffuse into the tissue to accumulate preferentially at the surface of nerve bundles in a localized area nearest to agent delivery patch 500.

FIG. 11 shows one embodiment of a method for using drug patch 500 to deliver an agent to the carotid body. Drug patch 500 may be placed on the side of the neck, in the vicinity of the angle of the mandible.

While the foregoing has been with reference to particular embodiments of the invention, it will be appreciated by those skilled in the art that changes in these embodiments may be made without departing from the principles and spirit of the invention.

1. A method for treating hypertension in a patient, the method comprising:
delivering a cardiac glycoside locally to a portion of a carotid body in an amount sufficient to impair function of the carotid body and lower a blood pressure of the patient.

2. method of claim 1, wherein the amount of the cardiac glycoside delivered is sufficient to reduce a nerve conductance in the portion of the carotid body.

3. The method of claim 1, wherein the amount of cardiac glycoside delivered is sufficient to affect chemosensors and/or chemoreceptors located in a vicinity of the carotid body.

4. The method of claim 1, wherein the amount of cardiac glycoside delivered is sufficient to affect a sympathetic tone within the patient.

5. The method of claim 1, wherein the amount of cardiac glycoside delivered is sufficient to normalize and restore a sympathetic balance, and/or affect a renal sympathetic nerve activity.

6. The method of claim 1, wherein the amount of the cardiac glycoside delivered is sufficient to induce death of nerve cells in the portion of the carotid body.

7. The method of claim 1, wherein the amount of the cardiac glycoside delivered is sufficient to induce death of nerve cells in the portion of the carotid body and prevent regrowth of nerve cells.

8. The method of claim 1, wherein the amount of the cardiac glycoside delivered is sufficient to impair nerve function by acting on an axonal segment of nerve cells in the portion of the carotid body.

9. The method of claim 1, wherein the amount of the cardiac glycoside delivered is sufficient to impair nerve function by inducing neuro-muscular block, sensory nerve block, or clinical nerve block.

10. The method of claim 1, wherein the amount of the cardiac glycoside delivered does not cause damage to tissue surrounding the carotid body.

11. The method of claim 1, wherein function of the carotid body is impaired temporarily.

12. The method of claim 1, wherein function of the carotid body is impaired for a sustained period of time.

13. The method of claim 1, wherein the cardiac glycoside is delivered in a time release formulation.

14. The method of claim 1, wherein the cardiac glycoside is digoxin.

15. The method of claim 1, wherein the amount of the cardiac glycoside delivered is approximately 0.2-1 mg/kg.

16. The method of claim 1, wherein the volume of the cardiac glycoside delivered is approximately 0.05-5 ml per administration.

17. The method of claim 1, wherein the amount of cardiac glycoside delivered is small enough and does not substantially enter the systemic circulation or cause organ damage.

18. The method of claim 1, wherein the amount of the cardiac glycoside delivered is sufficient to impair nerve function by acting on Schwann cells.

19. A method for treating a disease state in a patient, the method comprising:
accessing the carotid body directly with a syringe; and
injecting a neuromodulatory agent into the carotid body.

20. A method for treating a disease state in a patient, the method comprising:
placing a needle in a carotid body;
verifying a location of the needle using imaging;
using the needle to make an injection of a neuromodulatory agent into the carotid body; and
verifying the injection using imaging.

21-32. (canceled)
